(19) United States
(12) Patent Application Publication
(10) Pub. No.: US 2021/0137945 A1
(43) Pub. Date: May 13, 2021

(54) METHOD FOR TREATING A CANCER ASSOCIATED WITH THE ACTIVATION OF GALECTIN-1

(71) Applicant: TRINEO BIOTECHNOLOGY CO. LTD, New Taipei City (TW)

(72) Inventors: Teng-Hai CHEN, Tainan City (TW); Mon-Tarng CHEN, Tainan City (TW); Chien-Yuan WANG, Tainan City (TW); Cheng-Po HUANG, New Taipei City (TW); Su-Yu CHEN, New Taipei City (TW); Yi-Hsiu LIN, New Taipei City (TW); Ssu-Chia WANG, New Taipei City (TW); Chih-Yuan LIAO, New Taipei City (TW)

(73) Assignee: TRINEO BIOTECHNOLOGY CO. LTD, New Taipei City (TW)

(21) Appl. No.: 16/681,822

(22) Filed: Nov. 13, 2019

(51) Int. Cl. A61K 31/575; A61K 31/337; A61K 31/704; A61K 31/7048; A61P 35/00

(52) U.S. Cl. CPC A61K 31/575; A61K 31/337; A61P 35/00; A61K 31/7048; A61K 31/704

(57) ABSTRACT

Provided herein is a method for the treatment and/or prophylaxis of a cancer associated with galectin-1. The method includes administering to a subject a pharmaceutical composition that mainly composed of ganoderic acid S (GAS) and ganoderic acid T (GAT). The method further includes administering to the subject another anti-cancer agent before, together with, or after the administration of the present pharmaceutical composition, so as to synergistically suppress the growth of the cancer.

### BACKGROUND OF THE INVENTION

### 1. Field of the Invention

The present disclosure relates to a method for treating or preventing a cancer associated with activation of galectin-1 using the pharmaceutical composition comprising a novel combination of triterpenoids, ganoderic acid S and ganoderic acid T.

### 2. Description of Related Art

Galectin-1 has been implicated in cancer progression, invasion and metastasis. Evidence indicates that galectin-1 participates in tumor progression by evoking T cell energy and contributes to cancer-immune escape. Galectin-1 also relates to tumor endothelial cell adhesion and migration; therefore, Galectin-1 may act as a target for therapeutic intervention against cancer.

Inventors of the present disclosure unexpectedly identified certain ganoderic acids could suppress the progression and metastasis of a cancer mediated by the activation of galectin-1, thus these ganoderic acids may serve as candidates for the development of a medicament for the treatment of cancers associated with the activation of galectin-1.

### SUMMARY

The present disclosure is based, at least in part, unexpected discovery that certain triterpenoids or ganoderic acids isolated from the fruit bodies or mycelia of Ganoderma lucidum may suppress or inhibit the growth of cancerous cells associated with the activation of galectin-1. Thus, these compounds may serve as candidates for the development of a medicament for the treatment and/or prophylaxis of cancers associated with the activation of galectin-1.

Accordingly, it is the aspect of this disclosure to provide a method of treating or preventing a cancer associated with the activation of galectin-1 in a subject. The method includes the step of administering to the subject an effective amount of a pharmaceutical composition consisting essentially of ganoderic acid S (GAS) and ganoderic acid T (GAT); and a pharmaceutically acceptable excipient. According to embodiments of the present disclosure, the GAS and the GAT are present in the pharmaceutical composition in a weight ratio from about 1:1 to 1:10. Preferably, the GAS and GAT are present in the pharmaceutical composition in the weight ratio of 1:2.

The cancer associated with galectin-1 treatable by the present method may be any of glioblastoma, thyroid cancer, gastrointestinal cancer, liver cancer, lung cancer, breast cancer, pancreatic cancer, melanoma, prostate cancer, ovarian cancer, adenocarcinoma, bladder cancer and kidney cancer.

Alternatively or optionally, the method further includes the step of administrating to the subject an anti-cancer drug before, together with, or after the administration of the present pharmaceutical composition, so as to synergistically suppress the growth of the cancer.

Examples of the anti-cancer drug suitable for use in the present method include, but are not limited to, paclitaxel, docetaxel, camptothecin (CPT), topotecan (TPT), irinotecan (CPT-11), doxorubicin, daunorubicin, epirubicin, fluorouracil, cis-platin, cyclophosphamide, vinblastine, vincristine, ifosfamide, melphalan, mitomycin, methotrexate, mitoxantrone, teniposide, etoposide, bleomycin, leucovorin, cytarabine, dactinomycin, streptozocin, combretastatin A4-phosphate, and SU5416. In one preferred embodiment, synergistic suppression of the ovarian cancer is achieved when the present pharmaceutical composition is administered together with the anti-cancer drug of paclitaxel. In another preferred embodiment, synergistic suppression of the ovarian cancer is achieved when the present pharmaceutical composition is administered together with the anti-cancer drug of doxorubicin.

Alternatively or optionally, the method further includes the step of subjecting the subject to another treatment that is any of a surgical operation or radiation treatment before, concurrently with or after the step of administering the pharmaceutical composition of this disclosure to the subject.

The details of one or more embodiments of this disclosure are set forth in the accompanying description below. Other features and advantages of the invention will be apparent from the detail descriptions, and from claims.

It is to be understood that both the foregoing general description and the following detailed description are by examples, and are intended to provide further explanation of the invention as claimed.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present description will be better understood from the following detailed description read in light of the accompanying drawings, where:

FIG. 1 illustrates the effects of TN-GL-01 composition of present invention on ovarian cancer ES-2 cells in accordance with one embodiment of present invention;

FIG. 2 illustrates the effects of TN-GL-01 composition on the expression of galectin-1 via western blot analysis in accordance with one embodiment of present invention;

FIG. 3 illustrates the respective effects of TN-GL-01 composition, cisplatin, and doxorubicin on paclitaxel-induced cell death in accordance with one embodiment of present invention;

FIG. 4 illustrates the respective effects of TN-GL-01 composition, cisplatin, and doxorubicin on doxorubicin-induced cell death in accordance with one embodiment of present invention;

FIG. 5A are photographs illustrating the results of immunohistochemical analysis and the tumor appearances in accordance with one embodiment of present invention;

FIG. 5B is a bar graph depicting the weight of tumors of FIG. 5B;

FIG. 6A are photographs illustrating the results of immunohistochemical analysis on CD8-positive cells in accordance with one embodiment of present invention;

FIG. 6B is a bar graph depicting the percentage of human immune cells in the cancerous tissues in accordance with one embodiment of present invention;

FIG. 7A is a bar graph depicting the amount of paclitaxel retained in the cancerous tissues in accordance with one embodiment of present invention; and

FIG. 7B is a bar graph depicting the weight of tumors of FIG. 7A.

### DETAILED DESCRIPTION OF THE INVENTION

The detailed description provided below in connection with the appended drawings is intended as a description of the present disclosure and is not intended to represent the only forms in which the present disclosure may be constructed or utilized.

### 1. Definition

For convenience, certain terms employed in the context of the present disclosure are collected here. Unless defined otherwise, all technical and scientific terms used herein have the same meaning as commonly understood by one of the ordinary skill in the art to which this invention belongs.

The terms “treatment” and “treating” are used herein to include preventative (e.g., prophylactic), curative, or palliative treatment that results in a desired pharmaceutical and/or physiological effect. Preferably, the effect is therapeutic in terms of partially or completely curing or preventing the growth of tumor cells. Also, the term “treating” as used herein refers to application or administration of the pharmaceutical composition of the present disclosure to a subject, who has a medical condition, a symptom of the condition, a disease or disorder secondary to the condition, or a predisposition toward the condition, with the purpose to partially or completely alleviate, ameliorate, relieve, delay onset of, inhibit progression of, reduce severity of, and/or reduce incidence of one or more symptoms or features of a particular disease, disorder, and/or condition. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. As used herein, the symptom, disease, disorder or condition may be solid tumor or metastatic tumor. Treatment is generally “effective” if one or more symptoms or clinical markers are reduced as that term is defined herein.

The term “prophylaxis” as used herein means prevention against a future event. In the context of prophylaxis against tumor or tumor metastasis that may potentially occur as a consequence of a surgical or diagnostic procedure, the prophylactic administration can occur before, contemporaneous with, and/or after the procedure.

The term “an effective amount” as used herein refers to an amount effective, at dosages, and for periods of time necessary, to achieve the therapeutically desired result with respect to the treatment of cancer.

The terms “compounds,” “compositions,” “agent” and “medicament” are used interchangeably herein to refer to a compound or a composition of which, when administered to a subject such as a human or an animal induces a desired pharmacological and/or physiological effect by local and/or systemic action.

The terms “administered,” “administering” and “administration” are used interchangeably herein to refer means either directly administering a compound or a composition of the present invention, or administering a prodrug, derivative or analog which will form an equivalent amount of the active compound within the body.

The term “subject” or “patient” refers to an animal including the human species that is treatable with the compositions and/or methods of the present invention. The term “subject” or “patient” intended to refer to both the male and female gender unless one gender is specifically indicated. Accordingly, the term “subject” or “patient” includes, but is not limited to, human, non-human primate such as any mammal, dog, cat, horse, sheep, pig, cow, and etc., preferably a human, which may benefit from treatment by the compound of this disclosure. The terms “subject” and “patient” are used interchangeably in the present disclosure.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements. Also, as used herein, the term “about” generally means within 10%, 5%, 1%, or 0.5% of a given value or range. Alternatively, the term “about” means within an acceptable standard error of the mean when considered by one of ordinary skill in the art. Other than in the operating/working examples, or unless otherwise expressly specified, all of the numerical ranges, amounts, values and percentages such as those for quantities of materials, durations of times, temperatures, operating conditions, ratios of amounts, and the likes thereof disclosed herein should be understood as modified in all instances by the term “about.” Accordingly, unless indicated to the contrary, the numerical parameters set forth in the present disclosure and attached claims are approximations that can vary as desired. At the very least, each numerical parameter should at least be construed in light of the number of reported significant digits and by applying ordinary rounding techniques.

### 2. The Pharmaceutical Composition

The present disclosure is based, at least in part, unexpected discovery that a novel combination of certain ganoderic acids isolated from the fruit bodies or mycelia of Ganoderma lucidum, may suppress or inhibit the expression of galectin-1, which in term, leads to the suppression of the tumor. Accordingly, the novel combination of the identified ganoderic acids, particularly, the combination of ganoderic acid S (GAS) and ganoderic acid T (GAT), is useful as a therapeutic medicament for the treatment or prophylaxis of cancers mediated by galectin-1. More unexpectedly, a synergistic suppression of the growth and/or metastasis of a tumor is achieved when the present combination of GAS and GAT is administered with an anti-cancer drug, such as etoposide, doxorubicin, and paclitaxel.

Accordingly, it is the first aspect of this disclosure to provide a pharmaceutical composition for the treatment or prophylaxis of a cancer associated with the activation of galectin-1. The pharmaceutical composition includes a triterpenoid that consists essentially of ganoderic acid S (GAS) and ganoderic acid T (GAT); and a pharmaceutically acceptable excipient.

In general, GAS and GAT may be respectively isolated from Ganoderma spp in accordance with any method known in the art, such as from the fruit bodies of Ganoderma lucidum in accordance with the method described by Hirotani et al (Phytochemistry (1987), 26(10), 2797-2803). Alternatively, GAS and GAT may be isolated from the cultivating waste of Ganoderma lucidum, particularly from the mycelium remained in the cultivating media of Ganoderma lucidum after harvest, in accordance with the method described in Taiwan Patent No. 1381844, the disclosure of which is incorporated herein by reference. The isolation in general involves extracting the Ganoderma spp with a solvent, preferably an alcoholic solution, at a temperature above room temperature; followed by column chromatography purification, which may be high performance liquid chromatography (HPLC), reverse phase liquid chromatography and etc.; concentrating and drying the isolated product, until a dried powder is produced.

According to embodiments of the present disclosure, the GAS and the GAT are respectively present in the pharmaceutical composition in a ratio from about 1:1 to 1:10 by weight, such as 1:1, 1:2, 1:3, 1:4, 1:5, 1:6, 1:7, 1:8, 1:9, and 1:10 by weight. In one preferred embodiment, the GAS and the GAT exist in the pharmaceutical composition in a ratio of about 1:2 by weight.

The triterpenoid of the present disclosure, i.e., the combination of GAS and GAT, is present at a level of about 0.1% to 99% by weight, based on the total weight of the pharmaceutical composition, such as about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% by weight in the pharmaceutical composition. In some embodiments, the triterpenoid of the present disclosure is present at a level of at least 1% by weight, based on the total weight of the pharmaceutical composition. In certain embodiments, the triterpenoid of the present disclosure is present at a level of at least 5% by weight, based on the total weight of the pharmaceutical composition. In still other embodiments, the triterpenoid of the present disclosure is present at a level of at least 10% by weight, based on the total weight of the pharmaceutical composition. In still yet other embodiments, the triterpenoid is present at a level of at least 25% by weight, based on the total weight of the pharmaceutical composition.

In some embodiments, the medicament or the pharmaceutical composition of the present disclosure is used as an adjuvant therapy, in addition to the major cancer therapy, which includes, but is not limited to, surgical operation, radiotherapy, or chemotherapy. In some embodiments, the present pharmaceutical composition is used together with a chemotherapeutic agent or an anti-cancer agent.

The medicament or said pharmaceutical composition is prepared in accordance with acceptable pharmaceutical procedures, such as described in Remington's Pharmaceutical Sciences, 17^{th} edition, ed. Alfonoso R. Gennaro, Mack Publishing Company, Easton, Pa. (1985). Pharmaceutically acceptable excipients are those that are compatible with other ingredients in the formulation and biologically acceptable.

The pharmaceutical composition of the present disclosure may be administered by any suitable route, for example, orally in capsules, suspensions or tablets or by parenteral administration. Parenteral administration can include, for example, systemic administration such as intramuscular, intravenous, subcutaneous, or intraperitoneal injection. In preferred embodiments, the pharmaceutical composition of the present disclosure are administered orally (e.g., dietary) to the subject.

For oral administration, the pharmaceutical composition of the present disclosure may be formulated into tablets containing various excipients such as microcrystalline cellulose, sodium citrate, calcium carbonate, dicalcium phosphate, and glycine; along with various disintegrants such as starch, alginic acid and certain silicates; together with granulation binders like polyvinylpyrrolidone, sucrose, gelatin and acacia. Additionally, lubricating agents such as magnesium stearate, sodium lauryl sulfate and talc may be added. Solid composition may also be employed as fillers in gelatin capsules; preferred materials in this connection also include lactose or milk sugar as well as high molecular weight polyethylene glycols. When aqueous suspensions and/or elixirs are desired for oral administration, the active ingredient may be combined with various sweetening or flavoring agents, coloring matter or dyes, and if so desired, emulsifying and/or suspending agents as well, together with diluents such as water, ethanol, propylene glycol, glycerin and a combination thereof.

For parenteral administration, the medicament or pharmaceutical composition of the present disclosure may be formulated into liquid pharmaceutical compositions, which are sterile solutions, or suspensions that can be administered by, for example, intravenous, intramuscular, subcutaneous, or intraperitoneal injection. Suitable diluents or solvent for manufacturing sterile injectable solution or suspension include, but are not limited to, 1,3-butanediol, mannitol, water, Ringer's solution, and isotonic sodium chloride solution. Fatty acids, such as oleic acid and its glyceride derivatives are also useful for preparing injectables, as are natural pharmaceutically acceptable oils, such as olive oil or castor oil. These oil solutions or suspensions may also contain alcohol diluent or carboxymethyl cellulose or similar dispersing agents. Other commonly used surfactants such as Tweens or Spans or other similar emulsifying agents or bioavailability enhancers that are commonly used in manufacturing pharmaceutically acceptable dosage forms can also be used for the purpose of formulation.

It will be appreciated that the dosage of the pharmaceutical composition of the present disclosure will vary from patient to patient not only for the particular route of administration, and the ability of the composition to elicit a desired response in the patient, but also factors such as disease state or severity of the condition to be alleviated, age, sex, weight of the patient, the state of being of the patient, and the severity of the pathological condition being treated, concurrent medication or special diets then being followed by the patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the composition are outweighed by the beneficial effects. Preferably, the compositions of the present disclosure are administered at a dosage and for a time such that the number and/or severity of the symptoms are decreased.

### 3. The Treatment Method

The present disclosure also provides a method of treating a cancer associated with the activation of galectin-1 in a subject. The method includes the step of administering to the subject an effective amount of the pharmaceutical composition described above.

Alternatively or optionally, the method further includes the step of administering another agent known to improve the treatment of cancer, before, together with and/or after administering the pharmaceutical composition of this invention. Examples of such agent include, but are not limited to, anti-cancer drug, anti-angiogenesis agent, anti-virus agent, analgesic, anti-anemia drug, cytokine, granulocyte colony-stimulating factor (G-CSF), and anti-nausea drug and the like.

In some preferred embodiments, the method further includes administering to the subject an anti-cancer drug in addition to the administration of the present pharmaceutical composition, in which a synergistic effect on the suppression of cancers is achieved via suppressing the expression of galectin-1. Examples of anti-cancer drug suitable for use in the present method include, but are not limited to, paclitaxel, docetaxel, camptothecin (CPT), topotecan (TPT), irinotecan (CPT-11), Doxorubicin, daunorubicin, epirubicin, fluorouracil, cis-platin, cyclophosphamide, vinblastine, vincristine, ifosfamide, melphalan, mitomycin, methotrexate, mitoxantrone, teniposide, etoposide, bleomycin, leucovorin, cytarabine, dactinomycin, streptozocin, combretastatin A4-phosphate, SU5416, and the like. Examples of anti-angiogenesis agent include, but are not limited to, DS 4152, TNP-470, SU6668, endostatin, 2-methoxyestradiol, angiostatin, thalidomide, tetrathiomolybdate, linomide, IL-12, and the like. Examples of anti-virus agent include, but are not limited to, amantadine, rimantadine, and the like. Examples of analgesic include, but are not limited to, paracetamol such as para-acetylaminophenol, non-steroidal anti-inflammatory drug (NSAID) such as salicylates, and opioid drugs such as morphine and opium. Example of anti-anemia drug includes, but is not limited to, erythropoietin

Examples of cancers treatable by the present method include, but are not limited to, glioblastoma, thyroid cancer, gastrointestinal cancer, liver cancer, lung cancer, breast cancer, pancreatic cancer, melanoma, prostate cancer, ovarian cancer, adenocarcinoma, bladder cancer and kidney cancer.

In one preferred embodiment, the pharmaceutical composition is administered together with paclitaxel to a subject suffering from ovarian cancer. In another preferred the pharmaceutical composition is administered together with doxorubicin to a subject suffering from ovarian cancer.

In the present method, the pharmaceutical composition described above is administered to the subject in an amount of about 1 to 120 mg/Kg body weight of the subject per day, such as 1, 10, 20, 30, 40, 50, 60, 70, 80, 90, 100, 110 or 120 mg/Kg/day; preferably, in the amount of about 30 to 110 mg/Kg/day, such as 30, 40, 50, 60, 70, 80, 90, 100 or 110 mg/Kg/day; more preferably, in the amount of 80 to 100 mg/Kg/day, such as 80, 90, or 100 mg/Kg/day. It will be appreciated that the dosage of the pharmaceutical composition of the present disclosure will vary from patient to patient not only for the particular route of administration, and the ability of the composition to elicit a desired response in the patient, but also factors such as disease state or severity of the condition to be alleviated, age, sex, weight of the patient, the state of being of the patient, and the severity of the pathological condition being treated, concurrent medication or special diets then being followed by the patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the composition are outweighed by the therapeutically beneficial effects. The dose can be administered in a single dosage, or alternatively in more than one dosage, such as 2, 3, or 4 dosages a day.

In some embodiments, the method further includes subjecting the subject to a radiation treatment after the administration of the pharmaceutical composition of present invention.

The present invention will now be described more specifically with reference to the following embodiments, which are provided for the purpose of demonstration rather than limitation.

### Examples

### Materials and Methods

Cell Culture

Ovarian cancer cell ES-2 and myeloma cell line ARH-77 were used in the present disclosure. Cells was cultured and maintained in Dulbecco's modified Eagle media (DMEM) supplemented with 10% fetal bovine serum (FBS), 100 IU/ml penicillin, 100 ng/ml streptomycin, 2 mM glutamine, non-essential amino acids and sodium pyruvate in 5% CO₂ at 37° C. Cells were grown and maintained in Petri dishes (each was 10 cm in diameter) until they reached 80% confluence, and then were subject to cell passages. Briefly, cells were first washed with phosphate buffer solution (PBS, 3 ml) once, then treated with 0.05% Trypsin/0.025% EDTA solution (1 mL) for 5 min so that the attached cells become suspended. The suspended cells were harvested and 2 mL fresh culture media were added therein to neutralize any remaining activity of trypsin. Cell density was adjusted by adding appropriate amounts of culture medium to the cell suspension, which was then used to seed the culture plates. The plates were then returned to the incubator, and cultured in accordance with the steps described above.

Cell Activity Analysis

Cells were seeded in 96-well plates with a density of 3,000 cells/well and cultured in accordance with the procedures described above. On the day when cell activity analysis was to be conducted, cells were first treated with various concentrations of the triterpenoids of the present disclosure, imatinib or gefitinib for at least 48 hrs, before subjecting them to MTT assay.

MTT Assay

50 μL of the MTT stock solution (5 mg MTT dissolved in 1 ml of sterile PBS) was added to each well, and 50 μL of the MTT stock solution was added to 500 μL of medium alone and used as a negative control. Each sample was incubated at 37° C. for 4 hours. Aliquots (450 μl) from each sample were taken to a new well of 48-well culture plate, adding 1004, DMSO, mixing thoroughly using the pipette and reacted at 37° C. for 20 min and absorbance was measured at 570 nm.

Immunohistochemical Analysis

Tissues were fixed by 4% paraformaldehyde, embedded in paraffin. Tissue sections of ES2 tumor were processed for antigen retrieval. The samples were incubated using Ku80 (C48E7) rabbit monoclonal antibody, following by incubation with the anti-rabbit HRP secondary antibody. Samples were stained using DAB (brown) and counterstained with hematoxylin (blue).

Western Blot Analysis

Samples were respectively subjected to SDS-PAGE, transferred to PVDF membranes. These blots were blocked with 5% nonfat milk powder in TBS-0.1% Tween-20 for 30 minutes, followed by incubation with primary antibodies at 4° C. overnight and then horseradish peroxidase-conjugated secondary antibodies (Amersham Biosciences, Piscataway). Imaging of bands was performed using Pierce ECL Western Blotting Substrate (Thermo Fisher Scientific) and ImageQuant LAS-4000 (GE Healthcare).

Animal Model of Primary Ovarium Tumor

Male NOD/SCID mice about 6 weeks old were used in this experiment. Animals were kept at specific pathogen-free conditions under 12:12 light-dark cycle with food (laboratory rodent diet 5001 purchased from PMI Nutrition International Inc. MO, USA) and water (i.e., distilled water) provided ad libitum, ambient temperature and relative humidity were respectively set at 22±3° C. and 50±20%. All procedures involving animal studies of the present disclosure comply with the “Guideline for the Care and Use of Laboratory Animals” issued by The Chinese-Taipei Society of Laboratory Animal Sciences.

The day before tumor inoculation, mice were randomly divided into groups while making sure that the average weight in each group did not differ significantly. The fur on the right hind leg of each animal was then shaved. To inoculate tumor in mice, ovarian cancer cells ES-2 (1×10⁷ cancer cells/0.1 mL) were injected subcutaneously to NOD/SCID mice, to generate subcutaneous (s.c.) xenograft tumors.

### Example 1 Preparation and Characterization of the Present Pharmaceutical Composition

1.1 Preparation of the Present Pharmaceutical Composition

A pharmaceutical composition, herein “TN-GL-01 composition” was prepared by mixing the dried powders of GAT (26 mg), GAS (14 mg) and crystallized methylcellulose (MC) (60 mg) in a blender until a homogeneous mixture was obtained.

1.2 TN-GL-01 Composition Inhibited the Activity of Orarian Cancer Cells

The effects of the TN-GL-01 composition of example 1.1 on ovarian cancer cells (i.e., ES-2 cells) were evaluated by MTT assay in accordance with procedures described in the “Material and Methods” section. Results are provided in FIG. 1.

As depicted, 50% inhibition of ES-2 cells by the TN-GL-01 composition, GAS and GAT (IC₅₀) were independently 30.1, 58.3 and 24.5 μg/mL. Thus, as expected, TN-GL-01 composition of example 1.1, GAS, and GAT all exhibited cytotoxic effect on ES-2 cells.

1.3 TN-GL-01 Composition Inhibited Galectin-1 Expression of ES-2 Cells

In this example, the effects of TN-GL-01 composition on the expression of galectin-1 in ERK (extracellular-signal-regulated kinase) pathway was investigated via western blot analysis. Results are shown in FIG. 2. As depicted, GAS, GAT, and TN-GL-01 composition were all effective in suppressing the expression of galectin-1. Thus, it was concluded that the present TN-GL-01 composition exerted its function through suppression of galectin-1.

### Example 2 In Vitro Suppression of Cancer Cells by the Combined Treatment of TN-GL-01 Composition and an Anti-Cancer Drug

In this example, the effect of TN-GL-01 composition of example 1.1 on the cell killing effect of a chemotherapeutic drug (i.e., etoposide, doxorubicin, cisplatin, and paclitaxel) on myeloma ARH-77 cells was investigated, in which cyclophosphamide served as a comparative drug. Results are summarized in Tables 1 to 3, and FIGS. 3 and 4.

Reference is first made to Table 1, in which the data indicated that TN-GL-01 composition was more effective than cyclophosphamide in enhancing the etoposide induced cell death in myeoloma ARH-77 cells (IC₅₀ 156 nM vs 238 nM, compared to the control IC₅₀ of 415 nM). Similar results are also observed for another chemotherapeutic drug—doxorubicin (IC₅₀ 50 nM vs 73 nM, compared to the control IC₅₀ of 99 nM)

TABLE 1
IC₅₀ of myeoloma ARH-77 cells treated with TN-
GL-01 composition and a chemotherapeutic agent

| Treatment | IC₅₀ (nM) |
|---|---|
| Etoposide | 415 |
| Etoposide + Cyclophosphamide (3 mM) | 238 |
| Etoposide + TN-GL-01 (15 μg/mL) | 156 |
| Doxorubicin | 99 |
| Doxorubicin + Cyclophosphamide (3 mM) | 73 |
| Doxorubicin + TN-GL-01 (15 μg/mL) | 50 |

To determine the effects of combined use of TN-GL-01 and any other three drugs including paclitaxel, doxorubicin, and cisplatin, the statistical differences between expected value and observed value of the survival rate were calculated. If no significance existed between the expected and observed values, the effect is termed“additivity (p>0.05)”. If the observed value of the survival rate was significantly lower than the expected value, the given effect in combination is termed “synergy (p<0.05)”. Additionally, antagonism indicates that a statistically higher observed value of survival rate was found when comparing to its expected value of survival rate. The expected value was determined by multiplying the survival rate of each drug in specific dose. The obtained value and value of each survival rate were measured by MMT assay (FIG. 3 and FIG. 4). The results were presented as mean±standard deviation (SD) as shown in Tables 2 and 3.

Referring to FIG. 3, which depicts the respective effects of TN-GL-01 composition, cisplatin, and doxorubicin on paclitaxel induced cell death. Among the three anti-cancer agents (i.e., TN-GL-01 composition, cisplatin, and doxorubicin), TN-GL-01 composition was most effective in enhancing the paclitaxel induced cell death (FIG. 3). Surprisingly, synergistic suppression of the ARH-77 cells was observed when cells were treated with the combination of TN-GL-01 composition (20 μg/mL) and paclitaxel (62.5 nM and 125 nM, respectively) (see Table 2).

Similarly, TN-GL-01 composition was also most effective in enhancing the doxorubicin-induced cell death, as compared to that of cisplatin or paclitaxel (FIG. 4). Also, synergistic suppression of the ARH-77 cells was observed when cells were treated with the combination of TN-GL-01 composition (20 μg/mL) and doxorubicin at the concentration up to 500 nM (Table 3), whereas in the case when the concentration of doxorubicin was no more than 200 nM, then only additive or antagonic effect was observed (Table 3).

TABLE 2

| | Cell Survival Rate | | |
|---|---|---|---|
| Combination | Expected value | observed value | result |
| Paclitaxel (62.5 nM) + | 0.68 × 0.82 = | 0.38 ± 0.02 | Synergy |
| TN-GL-01 (20 μg/mL) | 0.56 ± 0.03 | (p = 0.002) | |
| Paclitaxel (125 nM) + | 0.50 × 0.82 = | 0.20 ± 0.03 | Synergy |
| TN-GL-01 (20 μg/mL) | 0.41 ± 0.03 | (p = 0.0008) | |
| Paclitaxel (62.5 nM) + | 0.68 × 0.58 = | 0.43 ± 0.03 | Additivity |
| Doxorubicin (100 nM) | 0.39 ± 0.02 | (p = 0.18) | |
| Paclitaxel (125 nM) + | 0.50 × 0.58 = | 0.38 ± 0.01 | Antagonism |
| Doxorubicin (100 nM) | 0.29 ± 0.01 | (p = 0.003) | |
| Paclitaxel (62.5 nM) + | 0.68 × 0.65 = | 0.48 ± 0.04 | Additivity |
| Cisplatin (3 μM) | 0.44 ± 0.04 | (p = 0.27) | |
| Paclitaxel (125 nM) + | 0.50 × 0.65 = | 0.34 ± 0.01 | Additivity |
| Cisplatin (3 μM) | 0.33 ± 0.03 | (p = 0.45) | |

TABLE 3

| | Cell Survival Rate | | |
|---|---|---|---|
| combination | Expected value | Observed value | result |
| Doxorubicin (250 nM) + | 0.66 × 0.68 = | 0.46 ± 0.02 | Additivity |
| TN-GL-01 (20 μg/mL) | 0.44 ± 0.02 | (p = 0.46) | |
| Doxorubicin (500 nM) + | 0.66 × 0.68 = | 0.25 ± 0.01 | Synergy |
| TN-GL-01 (20 μg/mL) | 0.44 ± 0.03 | (p = 0.0004) | |
| Doxorubicin (250 nM) + | 0.66 × 0.65 = | 0.56 ± 0.01 | Antagonism |
| Paclitaxel (40 μM) | 0.43 ± 0.02 | (p = 0.0004) | |
| Doxorubicin (500 nM) + | 0.66 × 0.65 = | 0.60 ± 0.01 | Antagonism |
| Paclitaxel (40 μM) | 0.43 ± 0.02 | (p = 0.0003) | |
| Doxorubicin (250 nM) + | 0.66 × 0.69 = | 0.53 ± 0.04 | Antagonism |
| Cisplatin (3 μM) | 0.45 ± 0.01 | (p = 0.03) | |
| Doxorubicin (500 nM) + | 0.66 × 0.69 = | 0.47 ± 0.03 | Additivity |
| Cisplatin (3 μM) | 0.45 ± 0.02 | (p = 0.31) | |

Taken together, the results from this example suggested that in addition to its own cytotoxicity on cancerous cells, the present TN-GL-01 composition may result in synergistic suppression of cancerous cells when administered together with another chemotherapeutic agent, such as paclitaxel and doxorubicin.

### Example 3 In Vivo Suppression of Ovarian Tumor by the Combined Treatment of TN-GL-01 Composition and Paclitaxel

In this example, ovarian tumor was innoculated and inducted in mice in accordance with procedures described in the “Material and Methods” section. Mice were randomly assigned into different groups and treated with the TN-GL-01 composition of example 1.1 (300 mg/Kg), paclitaxel (10 mg/Kg) or a combination of paclitaxel (10 mg/Kg or 20 mg/Kg) and the TN-GL-01 composition (300 mg/Kg) via intraperitoneal injection. Mice were then sacrificed, the expression of galectin-1 in stromal cells, the amount of paclitaxel retained in the cancerous tissues, as well as the level of CD-8 positive cells in the test animals were measured. Results are shown in FIGS. 5 to 7.

By appearance, the growth of ovarian tumor excised from the test animals treated with the TN-GL-01 composition of example 1.1 was significantly suppressed, as the weight of tumor was 52% less than that of the control animals (FIGS. 5A and 5B). Further, treatment with TN-GL-01 composition reduced the levels of galectin-1 in stroma cells, which was consistent with the finding in example 1.3. As to the expression of α-SMA, the number of stromal cells positively expressed α-SMA was lower than that of the control (FIG. 5A), which suggested that the TN-GL-01 composition might have changed the tumor microenvironment that facilitated the infiltration of immunce cells, and was consistent with the finding depicted in FIGS. 6A and 6B, in which an increased number of immune cells (i.e., CD-8) was found in TN-GL-01 composition treated ovarian tumor.

It was also found that TN-GL-01 composition enhanced the amount of a chemotherapeutic agent (i.e, paclitaxel) retained in the tumor tissue. As depicted in FIG. 7A, the amount of paclitaxel remained in the tumor tissue was significantly higher in tumors treated with TN-GL-01 (300 mg/Kg) and paclitaxel (10 or 20 mg/Kg). Accordingly, the tumor weight was also significantly reduced in tumors treated with the combination of TN-GL-01 (300 mg/Kg) and paclitaxel (10 or 20 mg/Kg) (FIG. 7B).

Taken together, results of the present disclosure indicate that TN-GL-01 composition is suitable for the development of a medicament or an adjuvant for treating cancers.

It will be understood that the above description of embodiments is given by way of example only and that various modifications may be made by those with ordinary skill in the art. The above specification, examples and data provide a complete description of the structure and use of exemplary embodiments of the invention. Although various embodiments of the invention have been described above with a certain degree of particularity, or with reference to one or more individual embodiments, those with ordinary skill in the art could make numerous alterations to the disclosed embodiments without departing from the spirit or scope of the present disclosure.

What is claimed is:

1. A method of treating a cancer associated with the activation of galectin-1 in a subject comprising administering to the subject an effective amount of a pharmaceutical composition consisting essentially of ganoderic acid S (GAS) and ganoderic acid T (GAT); and a pharmaceutically acceptable excipient.

2. The method of claim 1, wherein the GAS and the GAT are present in the pharmaceutical composition in a weight ratio from about 1:1 to 1:10.

3. The method of claim 1, wherein the GAS and the GAT are present in the pharmaceutical composition in a weight ratio of about 1:2.

4. The method of claim 1, wherein the cancer associated with the activation of galectin-1 is selected from the group consisting of glioblastoma, thyroid cancer, gastrointestinal cancer, liver cancer, lung cancer, breast cancer, pancreatic cancer, melanoma, prostate cancer, ovarian cancer, adenocarcinoma, bladder cancer and kidney cancer.

5. The method of claim 4, wherein the cancer associated with the activation of galectin-1 is ovarian cancer.

6. The method of claim 1, further comprising administrating an anti-cancer drug to the subject.

7. The method of claim 6, wherein the anti-cancer drug is selected from the group consisting of paclitaxel, docetaxel, camptothecin (CPT), topotecan (TPT), irinotecan (CPT-11), doxorubicin, daunorubicin, epirubicin, fluorouracil, cisplatin, cyclophosphamide, vinblastine, vincristine, ifosfamide, melphalan, mitomycin, methotrexate, mitoxantrone, teniposide, etoposide, bleomycin, leucovorin, cytarabine, dactinomycin, streptozocin, combretastatin A4-phosphate, and SU5416.

8. The method of claim 7, wherein the anti-cancer drug is paclitaxel.

9. The method of claim 7, wherein the anti-cancer drug is doxorubicin.

10. The method of claim 7, wherein the anti-cancer drug is etoposide.
